(19) United States
(12) Patent Application Publication
(10) Pub. No.: US 2006/0228402 A1
(43) Pub. Date: Oct. 12, 2006

(54) Techniques for forming a lipid bilayer membrane

(75) Inventors: Elena Pohl, Muhlenbeck (DE); Valeri Beck, Berlin (DE)

Correspondence Address: SUGHRUE MION, PLLC, 2100 PENNSYLVANIA AVENUE, N.W., SUITE 800, WASHINGTON, DC, 20037, US

(73) Assignee: CHARITE-UNIVERSITATSMEDIZIN BERLIN

(21) Appl. No.: 11/101,550

(22) Filed: Apr. 8, 2005

(51) Int. Cl. B01L 3/02; A61K 9/127

(52) U.S. Cl. 424/450; 422/100

(57) ABSTRACT

A method for forming a lipid bilayer membrane is provided, the method comprising forming a layer of lipid molecules on a liquid surface at an air-liquid interface between a liquid volume and an air volume. An aperture is moved from the liquid volume to the air volume through the layer of lipid molecules to form a lipid monolayer membrane at the aperture, the aperture being at an end of bilayer former. The aperture is then moved from the air volume to the liquid volume through the layer of lipid molecules to form the bilayer membrane. An angle of >90° is maintained between the liquid surface and a surface of the aperture during the formation of the lipid monolayer membrane and during the formation of the lipid bilayer membrane.

This disclosure teaches techniques for forming a lipid bilayer membrane.

### BACKGROUND

The number of membrane proteins identified from genome sequencing and cDNA library searches now greatly exceeds the number of electrophysiologically characterized transport and channel proteins, and identification of their functions based on their transport properties remains a challenge for the future.

Whereas the investigation of small water-soluble proteins exhibits no essential problems, handling of highly hydrophobic membrane proteins still requires advances in methodological approach. A technique for evaluating electrophysiological properties of protein channels, a patch clamp recording of the cell membrane (see Sakmann, B. et al.: Annu. Rev. Physiol 46, 455-472 (1984)), provides good signal resolution, however, it is sometimes difficult to assign the measured channels to specific proteins. Moreover, it is inapplicable for studies of ion channels located on intercellular membranes, for example, in endoplasmic or sarcoplasmic reticulum.

Protein reconstitution into artificial lipid bilayers represents an alternative method allowing the biochemical analysis of defined protein preparations in a precisely known lipid and buffer environment. Since the first description of artificial planar lipid membrane formation (see Mueller, P. et al.: J. Phys. Chem. 67, 534-535 (1963)), two main approaches allowing successful reconstitution of isolated and purified protein have been developed: (i) a “monolayer elevation” method described (see Schindler, H. et al.: Methods Enzymol. 171, 225-253 (1989)) and (ii) membrane formation on glass patch pipettes (see Hanke, W. et al.: Biochim. Biophys. Acta 727, 108-114 (1983)). The first technique uses the finding that monolayers are formed spontaneously at the air-water interface of a proteoliposome suspension. Two monolayers can be combined into a protein-containing bilayer by raising the solution level on both sides of a septum that divides the chamber into two parts (see for example Schindler, H.: FEBS Lett. 122, 77-79 (1980)). At present, this method seems to be the most frequently used approach for studies of purified proteins in artificial lipid bilayers (Saparov, S. M. et al.: J. Biol. Chem. 276, 31515-31520 (2001)). Disadvantages of this technique include cleaning and preparation of the chamber, as well as hole punching.

The second method implies the formation of planar lipid bilayer membranes at the end of glass patch clamp pipettes. Better current resolution and simple handling have been claimed as advantages of this method. However, the use of this interesting approach appears to have declined in recent years. The reasons include a high membrane fragility and difficulties in control for the membrane quality because membrane capacity and, thus, membrane diameter cannot be measured accurately due to membrane movements inside the patch pipette. Both methods are rather time consuming and require a well-trained researcher.

### SUMMARY

Therefore, it is desirable to provide improved techniques for protein reconstitution into a lipid bilayer membrane and apparatus which can be used for an automated lipid bilayer membrane formation, especially for protein reconstitution into an lipid bilayer membrane.

According to an aspect of the disclosed teachings a method for forming a lipid bilayer membrane is provided, the method comprising:

a) forming a layer of lipid molecules on a liquid surface at an air-liquid interface between a liquid volume and an air volume;

b) moving an aperture from the liquid volume to the air volume through the layer of lipid molecules to form a lipid monolayer membrane at the aperture, the aperture being at an end of a bilayer former;

c) moving the aperture from the air volume to the liquid volume through the layer of lipid molecules to form the lipid bilayer membrane;

where an angle of >90° is maintained between the liquid surface and a surface of the aperture during the formation of the lipid monolayer membrane and during the formation of the lipid bilayer membrane.

Another aspect of the disclosed teachings is an apparatus for forming a lipid bilayer membrane, the apparatus comprising: a liquid reservoir operable to receive a liquid volume having an air-liquid interface between the liquid volume and an air volume, a liquid surface of the liquid volume having a layer of lipid molecules on the liquid surface;
a bilayer former with an aperture at one end;
a mover operable to move the bilayer former; and
a controller operable to control the moving of the bilayer former;
   where the bilayer former and the mover are operable to move said aperture from said liquid volume into said air volume through said layer of lipid molecules on said liquid surface, thereby forming a lipid monolayer membrane in said aperture and are further operable to move the aperture comprising said lipid monolayer membrane from said air volume into said liquid volume through said layer of lipid molecules on said liquid surface, thereby forming a lipid bilayer membrane in said aperture

Further enhancements are provided as listed in the dependent claims.

With the disclosed teachings, an automatic approach is provided which reduces substantially the efforts and allow the usage of one-way material for forming a lipid bilayer membrane, especially for the reconstitution of membrane proteins in artificial membranes.

For example, conventional pipettes with a diameter of about 150 μm to about 300 μm can preferably used as bilayer former. Because such membranes are much larger than membranes formed on the tip of a glass patch pipette (about 0.5 μm to 5 μm), much more protein can be reconstituted. As a result the signal to noise ratio is improved. Albeit the membrane surface is bigger, the amount of proteoliposomes is reduced because less buffer solution is required (about 50 to 100 μl). In contrast to the known glass pipette method, the specific membrane capacitance, C_{spec}, can be measured exactly, because the diameter of the membrane provided can be precisely measured by microscope. Thus, an important criterion for the verification of bilayer formation is fulfilled. An important advantage of the proposed apparatus is worth mentioning. Conventional plastic pipettes and container may be used which are cheap one-way objects and can be easily replaced. No additional expensive devices are necessary for pipette pulling.

### BRIEF DESCRIPTION OF THE DRAWINGS

The above objectives and advantages of the disclosed teachings will become more apparent by describing in detail preferred embodiments thereof with reference to the attached drawings which show as follows:

FIG. 1 a schematic view of an apparatus for automated protein reconstitution into lipid bilayer membranes;

FIG. 2A-2F a schematic showing the formation of solvent-free bilayers from a preformed lipid monolayer at an aperture of a pipette tip made of a plastic material;

FIG. 3 current-voltage relationships of UCP2-containing membranes in the absence (circles) or presence (triangles) of ω-6-eicosatrienoic acid (EA), and of membranes containing EA and 0.9 mM ATP (diamonds);

FIG. 4 gramicidin pore fluctuation in bilayers made from E. coli lipid in the aperture of the pipette tip, where a buffer solution contained 0.5 M KCl at pH 6.8; and

FIG. 5 a schematic view of an apparatus for automated protein reconstitution into lipid bilayer membranes in a multi-channel set-up.

### DETAILED DESCRIPTION OF THE EMBODIMENTS

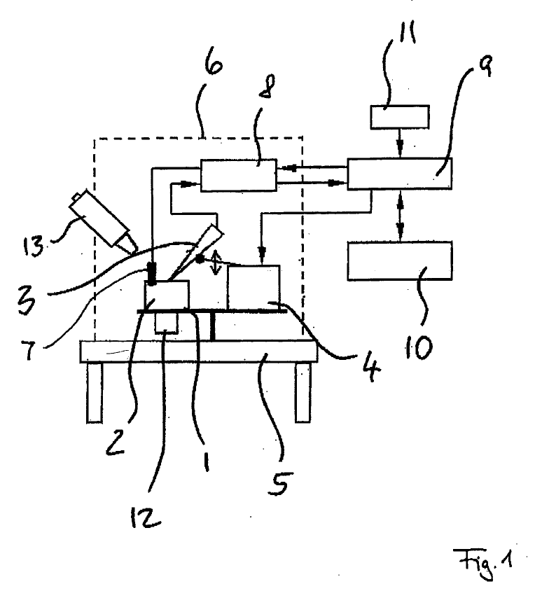

Referring to FIG. 1, an apparatus for forming a lipid bilayer membrane is depicted. A thermostatic unit 1 is provided with a container 2 for receiving a liquid volume. A pipette tip 3 is held by a pipette elevator 4 for moving the pipette tip 3 through a liquid surface of the liquid volume in the container 2. The pipette tip 3 is made of a plastic material. The container 2, and the pipette elevator 4 are located on a vibration isolation table 5. A faraday cage 6 is provided.

A reference electrode 7 is connected to a preamplifier 8. The preamplifier 8 is connected to an amplifier 9 with built-in AD converter. For controlling measures, a Personal Computer 10 is connected to the amplifier 9. In addition, for noise reduction, a power line conditioner 11 connected to the amplifier 9 is provided. The liquid in the container 2 can be stirred by means of a stirrer 12. A light microscope 13 is provided to observe the membrane area formed in the process.

Following, the use of the apparatus depicted in FIG. 1 for lipid membrane formation is described.

1. Formation of bilayer membranes from UCP2-containing monolayers.

For UCP2 expression, extraction, purification and reconstitution into liposomes human UCP2-containing plasmids were transformed into the bacterial strain BL21 (Novagen) as known from prior art (see Zackova, M. et al.: Biosci. Rep. 22, 33-46 (2002); Jaburek, M. et al.: J. Biol. Chem. 278, 25825-25831 (2003); Jaburek, M. et al.: J. Biol. Chem. 274, 26003-26007 (1999); Jaburek, M. et al.: J. Biol. Chem. 276, 31897-31905 (2001)). Extraction from inclusion bodies and purification of UCP2 followed previously described protocol. A minor modification included the type and the amount of phospholipids used, so that 2.5 mg of the total phospholipids extract from E. coli (Sigma) was used per mg of inclusion bodies.

The obtained sample of recombinant UCP2 was reconstituted into liposomes using previously described procedures, employing incubation of the protein/lipid mixture in 30 mM K-TES, 80 mM K₂SO₄, 2 mM EDTA, pH 7.2, with Bio-Beads SM-2 (Bio-Rad). The obtained proteoliposomes were stored at −80° C. and thawed the day of experiment. To introduce fatty acids, proteoliposomes were mixed with FA-containing liposomes in required proportions and unilamellar vesicles, containing FA and UCP2 were than extruded through the filter of 100 nm pore diameter 21 times using a small-volume extrusion apparatus (Avestin Inc., Ottawa, Canada).

Bilayer membranes containing UCP2 were formed on the pipette tip 3 (see FIG. 1) having a diameter of about 150 μm to about 300 μm.

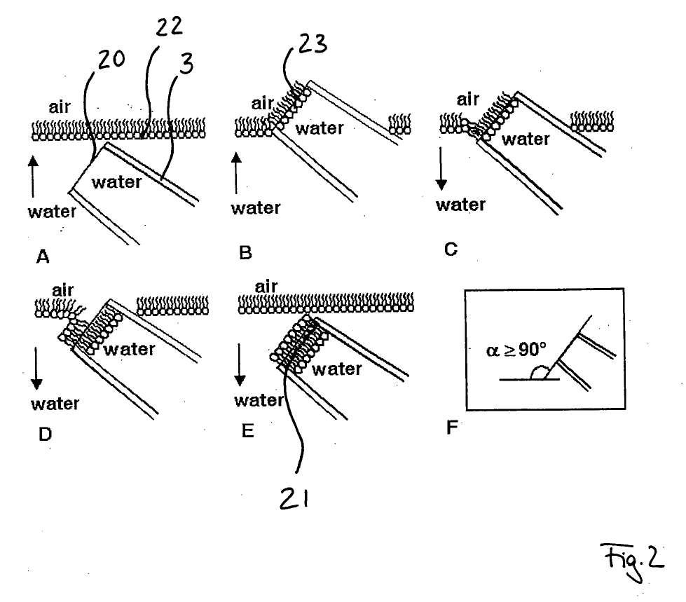

Referring to FIG. 2A-2F, at the beginning of the experiment, the pipette tip 3 was filled with the buffer solution and attached to the pipette elevator 4 manually. An angle >90° is maintained between the water surface and a pipette aperture surface 20 (see FIG. 2F). The pipette tip 3 was therefore flexed. A small plastic one-way container 2 with a volume of 0.75 ml and surface diameter of 12 mm was filled with the proteoliposome suspension, permanently stirred and maintained at 37° C. A bilayer 21 is formed by the transfer of the first one (see FIG. 2A, 2B) and then the second phospholipid monolayer (see FIG. 2C-2E) to the pipette tip 3. The pipette tip 3 and phospholipids are not drawn to scale.

The pipette tip 3 was placed several millimeters under a air-solution surface 22 (see FIG. 2A) and pipette elevator 4 (see FIG. 1) was started. The raising of the pipette tip 3 from the bath led usually to the formation of a first monolayer 23. After achieving a fix point over the air-solution surface 22, the pipette tip 3 was lowered slowly into the solution again. As the pipette tip 3 passes through the water-lipid interface, it becomes coated with a second monolayer of lipid molecules, thereby forming the lipid bilayer 21. Pipette tip 3 elevating and lowering was carried out automatically. Typically the device operated at a frequency of about 0.5 Hz to 1 Hz. It was controlled by an electronic controller, which stopped the up and down movement after membrane formation. Therefore a feedback loop was installed to the analog output of the current amplifier.

The reconstitution of purified recombinant uncoupling protein 2 (UCP2) was described above. The latter lacks the disadvantages of the prior art methods and allows the automatic formation of large membranes at the tip of a pipette. Urgent needs to study UCP2 was inspired by discoveries of a range of mitochondrial inner membrane proteins belonging to the uncoupling protein subfamily (UCP2, UCP3, UCP4, UCP5) by aimed searching of cDNA library. The novel proteins have a high homology to UCP1, which is expressed exclusively in brown adipose tissue. The distribution pattern of UCP2-UCP5 is surprising, reaching from UCP2 transcript, ubiquitously present of in all mammalian tissues, over the preferential distribution of UCP3 in muscles to the mainly brain expressed UCP4 and UCP5. Their molecular transport mechanisms and functions are either unknown or in dispute, despite numerous experiments using reconstituted recombinant proteins, cell cultures and knock out mice.

2. Monitoring the capacitance of the membrane, made of UCP2 containing liposomes.

To verify the formation of the bilayer 21 (see FIG. 2E), the capacity of the membrane was permanently monitored. For this goal, a triangle input wave with a peak to peak amplitude of 100 mV and a frequency of 10 Hz was applied to the input reference electrode after starting the pipette elevator 4 (see FIG. 1). An electronic scheme, connected to the amplifier 9, was analyzing the output signal from the reference electrode 7. A feedback signal was transmitted to the pipette elevator 4 allowing a continuous cycle of pipette elevation followed by slow pipette lowering if bilayer formation was not indicated by capacitance measurements. In the case of successful membrane formation, the feedback signal stopped the elevation process. Specific capacity, C_{spec}, was calculated with respect to membrane surface area as determined using the light microscope 13. It was equal to 0.95±0.01 μF/cm² for bilayers both with and without protein. These values are similar to the values reported for membranes formed from proteoliposomes. The measured capacity remained constant during the experiment.

3. Measurements of the membrane conductivity in presence of ω-6-eicosatrinoic acid.

Current-voltage (I-V) characteristics were measured by a patch-clamp amplifier (EPC 10, Heka Elektronik Dr. Schulze GmbH, Germany). For conductance measurements, a ramp voltage signal operating at frequencies of 0.016 Hz was used. Membrane conductance G was determined at zero voltage from a linear fit of voltages in the interval between −50 and 50 mV. For noise reduction the power-line conditioner 11 (see FIG. 1) was used. The apparatus was fixed onto the vibration-free table 5. Data acquisition and processing were performed by Software Pulse (v. 8.65, HEKA Elektronik Dr. Schulze GmbH, Germany).

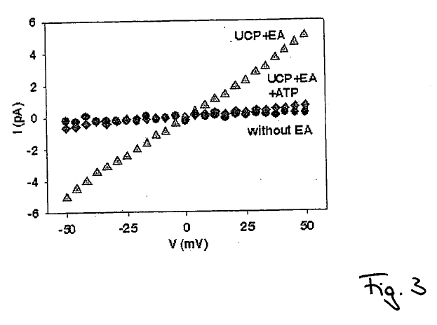

FIG. 3 shows current-voltage relationships of UCP2-containing membranes in the absence (circles) or presence (triangles) of ω-6-eicosatrienoic acid (EA), and of membranes containing EA and 0.9 mM ATP (diamonds). Lipid content was 1,5 mg/ml. The buffer solution contained 50 mM K₂SO₄, 25 mM TES, 0.6 mM EGTA, pH 7.35, and the temperature was 37° C.

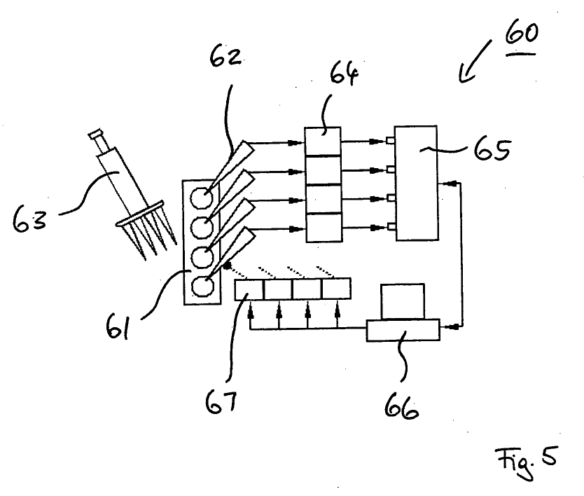

Analogously to previous results with UCP1, the increased UCP2-mediated conductivity in presence of FA was nearly completely inhibited by ATP, if added on both sides of the membrane. FIG. 5 shows results for the inhibition of UCP2-mediated FA-dependent proton transport by ATP. The concentration of UCP2 and ATP were 15 μg/ml and 0.1 mM, respectively

4. Reconstitution of the model peptide gramicidin.

In the apparatus (see FIG. 1), the peptide gramicidin (Fluka Chemie GmbH, Buchs, Switzerland) was added from an ethanolic stock solution to the buffer solution. G gramicidin is known to form characteristic voltage-dependent multi-level pore fluctuations in bilayer membranes (see Hladky, S. B. et al.: Nature 5231, 451-453 (1970); Hladky, S. B. et al.: Biochim. Biophys. Acta 274, 294-312 (1972); Bamberg, E. et al.: J. Membr. Biol. 11, 177-194 (1973)). Because gramicidin exhibits these characteristic channel states only in true bilayer membranes, their visulisation provides additional evidence that the pipette is sealed by a single lipid bilayer.

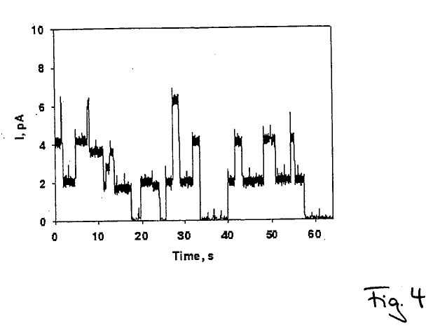

FIG. 4 illustrates the typical gramicidin channel activity in the apparatus described above. A solvent-free membrane monolayer was made by forming a monolayer from the total lipid E. coli extract (Sigma Chemical Co., St. Louis, Mo.) on top of the buffer solution. Buffer solution contained 0.5 M KCl at pH 6.8. Gramicidin was added at a concentration of 5*10⁻⁷ mg/ml. The holding potential was 100 mV, the temperature was 37° C. Current flow through the bilayer was monitored using the same amplifier (EPC 10, HEKA). The chamber was clamped at virtual ground while the pipette was clamped to the desired holding potential of 100 mV.

With the disclosed teachings an automatic approach is provided which reduces substantially the efforts and allow the usage of one-way material for forming a lipid bilayer membrane, especially for the reconstitution of membrane proteins in artificial membranes. In comparison to known reconstitution methods at least three important changes were made:

(i) Conventional pipettes with a diameter of about 150 μm to about 300 μm were used. Because such membranes are much larger than membranes formed on the tip of a glass patch pipette (about 0.5 μm to 5 μm), much more protein can be reconstituted. As a result the signal to noise ratio is improved. Albeit the membrane surface is bigger, the amount of proteoliposomes is reduced because less buffer solution is required (about 50 to 100 μl). In contrast to the glass pipette method, the specific membrane capacitance, C_{spec}, can be measured exactly, because the diameter of the membrane on the pipette tip can be precisely measured by microscope. Thus, a criterion for the verification of bilayer formation is fulfilled. An advantage of the proposed apparatus is worth mentioning. Conventional plastic pipettes and container are cheap one-way objects and can be easily replaced. No additional expensive devices are necessary for pipette pulling.

(ii) A critical moment in the membrane formation is the relative position of the pipette tip to the water surface. For successful membrane formation the angle between the bath and pipette surfaces was hold at an angle of >90° (see FIG. 2).

(iii) An advantage is the automation of the membrane formation process. As a consequence, the method is especially useful for an user having little experience with the model membrane techniques. The automatic approach allows future extensions to simultaneous formation of several independent membranes, for example, in well plates (see FIG. 5). Conceivably, it enables high throughput screening of ion channels and carriers. FIG. 5 shows a multi-channel set-up 60 comprising a set of devices which are similar to the one depicted in FIG. 1. A set of pipettes tips 62 with electrodes is provided each connected to an amplifier 64. The amplifiers 64 are connected to a respective AD converter 65. A pipette tip elevator 67 is used for raising and lowering each of the pipette tips 62 in a container 61. A Personal Computer 66 is used for operation control and/or data acquisition. There is also provided a multi-channel pipetor 63. Operation of the multi-channel set-up 60 in FIG. 5 is similar to operation of the apparatus according to FIG. 1 which is described in detail above.

Using the advantages of the disclosed teachings, it was established that an increase in the conductivity of UCP2-containing membranes occurs exclusively in the presence of FA such as eicosatrienoic acids (FIG. 3), therefore it can be concluded that FAs are essential for UCP2 function. These acids have previously been shown to activate UCP1 in bilayer membranes (see Urbankova, E. et al.: J. Biol. Chem. 278, 32497-32500 (2003)) and in proteoliposomes (see Zackova, M. et al.: J. Biol. Chem. (2003)).

The reports, concerning the activation of UCP2 by these FAs are contradictory. By ATP addition to the buffer solution on both sides of the membrane the UCP2-mediated conductivity was nearly completely inhibited in the range of studied voltages −50 to +50 mV. The similarity of activation and inhibition patterns of UCP2 to previously described results with UCP1 (see Urbankova, E. et al.: J. Biol. Chem. 278, 32497-32500 (2003)) demonstrates significantly that UCP2 as well might act as an uncoupler. However, to achieve a comparable increase in conductivity a 20-fold higher UCP2 concentration was required. This can be partly explained by not complete refolding of UCP2 from inclusion bodies. The lower uncoupling efficiency of UCP2 raises the question whether UCP2 can generate enough heat for thermogenesis (see Jezek, P. et al.: Physiol Res. 53 Suppl 1, S199-S211 (2004)). Conceivably, the amount of protons transported is able just to attenuate the mitochondrial production of reactive oxygen species. This ability makes this protein an important player in physiology and oxidative stress-related pathologies including atherosclerosis, ischemia-reperfusion damage, inflammation, type-2 diabetes, Parkinson's disease, Alzheimer's disease, and other neurodegenerative diseases.

The features disclosed in this specification, claims and/or the figures may be material for the realization of the invention in its various embodiments, taken in isolation or in various combinations thereof.

Other modifications and variations to the invention will be apparent to those skilled in the art from the foregoing disclosure and teachings. Thus, while only certain embodiments of the invention have been specifically described herein, it will be apparent that numerous modifications may be made thereto without departing from the spirit and scope of the invention.

What is claimed is:

1. A method of forming a lipid bilayer membrane, the method comprising:
a) forming a layer of lipid molecules on a liquid surface at an air-liquid interface between a liquid volume and an air volume;
b) moving an aperture from the liquid volume to the air volume through the layer of lipid molecules to form a lipid monolayer membrane at the aperture, the aperture being at an end of a bilayer former;
c) moving the aperture from the air volume to the liquid volume through the layer of lipid molecules to form the lipid bilayer membrane;
where an angle of >90° is maintained between the liquid surface and a surface of the aperture during the formation of the lipid monolayer membrane and during the formation of the lipid bilayer membrane.

2. Method according to claim 1, wherein the aperture is moved from the liquid volume into the air volume by elevating the aperture from the liquid volume into the air volume.

3. Method according to claim 1, wherein the aperture is moved from the air volume into the liquid volume by lowering the aperture from the air volume into the liquid volume.

4. Method according to claim 1, wherein a space provided inside the former in fluid connection with said aperture is filled with a solution prior to formation of said lipid monolayer membrane.

5. Method according to claim 1, wherein the formation of said lipid bilayer membrane is started when a lower end of the aperture reaches said air-liquid interface.

6. Method according to claim 1, wherein said formation of said lipid monolayer membrane and said formation of said lipid bilayer membrane are automatically controlled by a control unit.

7. Method according to claim 1, wherein said former is a pipette tip.

8. Method according to claim 1, wherein said former is made of a plastic material or glass.

9. Method according to claim 1, wherein said former is an one-way article.

10. Method according to claim 1, wherein a diameter of a cross section of the aperture is a range from 50 μm to 300 μm.

11. Method according to claim 1, wherein said formation of said lipid monolayer membrane and said formation of said lipid bilayer membrane are automatically repeated with a repetition rate of about 0.5 Hz to about 2 Hz.

12. An apparatus for forming a lipid bilayer membrane, the apparatus comprising:
a liquid reservoir operable to receive a liquid volume having an air-liquid interface between the liquid volume and an air volume, a liquid surface of the liquid volume having a layer of lipid molecules on the liquid surface;
a bilayer former with an aperture at one end;
a mover operable to move the bilayer former; and
a controller operable to control the moving of the bilayer former;
where the bilayer former and the mover are operable to move said aperture from said liquid volume into said air volume through said layer of lipid molecules on said liquid surface, thereby forming a lipid monolayer membrane in said aperture and are further operable to move the aperture comprising said lipid monolayer membrane from said air volume into said liquid volume through said layer of lipid molecules on said liquid surface, thereby forming a lipid bilayer membrane in said aperture.

13. Apparatus according to claim 12, wherein said mover is an elevator.

14. Apparatus according to claim 12, wherein the bilayer former is a pipette tip.

15. Apparatus according to claim 14, wherein said bilayer former is made of a plastic material or glass.

16. Apparatus according to claim 12, wherein said bilayer former is an one-way article.

17. Apparatus according to claim 12, wherein a diameter of a cross section of the aperture is in a range from 50 μm to about 300 μm.

18. Apparatus according to claim 12, wherein said controller is operable to automatically repeat said formation of said lipid monolayer membrane and said formation of said lipid bilayer membrane with a repetition rate of about 0.5 Hz to about 2 Hz

19. Apparatus according to claim 12, wherein said bilayer former is operable to maintain an angle of >90° between the liquid surface and a surface of said aperture during said formation of said lipid monolayer membrane and during said formation of said lipid bilayer membrane.

20. Apparatus according to claim 12, wherein a second bilayer former, the second bilayer former being connected to a second mover is provided.
